(19) United States
(12) Patent Application Publication
(10) Pub. No.: US 2012/0029046 A1
(43) Pub. Date: Feb. 2, 2012

(54) CRYSTALLINE FORM OF SUNITINIB AND PROCESSES FOR ITS PREPARATION

(75) Inventors: Vinayak Gore, Maharashtra (IN); Bharati Choudhari, Maharashtra (IN); Mahesh Hublikar, Maharashtra (IN); Prakash Bansode, Maharashtra (IN)

(73) Assignee: Generics (UK) Limited, Hertfordshire (GB)

(21) Appl. No.: 13/060,331

(22) PCT Filed: Aug. 24, 2009
(86) PCT No.: PCT/GB09/51054

§ 371 Date: Jul. 12, 2011

(30) Foreign Application Priority Data
Aug. 25, 2008 (IN) 1434/KOL/2008

(51) Int. Cl. A61K 31/404; A61P 35/00; C07D 403/06

(52) U.S. Cl. 514/414; 548/468

(57) ABSTRACT

The present invention relates to a novel crystalline form of sunitinib free base designated form I and to processes for its preparation. The invention also relates to its use as an API and in the preparation of various forms of sunitinib. Further, the invention relates to pharmaceutical compositions comprising the novel crystalline form and salts, solvates and hydrates prepared according to the invention, and to the uses of said pharmaceutical compositions in the treatment and/or prevention of cancer.

### FIELD OF THE INVENTION

The present invention relates to a novel crystalline form of sunitinib free base designated form I and to processes for its preparation. The invention also relates to its use as an API and in the preparation of various forms of sunitinib. Further, the invention relates to pharmaceutical compositions comprising the novel crystalline form and salts, solvates and hydrates prepared according to the invention, and to the uses of said pharmaceutical compositions in the treatment and/or prevention of cancer.

### BACKGROUND OF THE INVENTION

Sunitinib, represented by formula (I) and chemically named N[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide, is an oral tyrosine kinase inhibitor (TKI) that targets and blocks the signaling pathways of multiple selected receptor tyrosine kinases (RTKs).

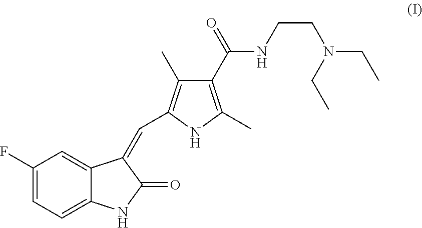

Through competitive inhibition of ATP binding sites, sunitinib inhibits the TK activity of a group of closely related RTKs, all of which ate involved in various human malignancies: the vascular endothelial growth factor receptors (VEGFR-1, -2, -3), the platelet derived growth factor receptors (PDGF-R), the stem cell factor (KIT), CSF-1R, Flt3, and RET. Sunitinib is therefore useful for the treatment of cancer and tumors. It is currently marketed for the treatment of unresectable and/or metastatic malignant gastrointestinal stromal tumor (GIST) and advanced and/or metastatic renal cell carcinoma (MRCC). The product is marketed as sunitinib malate under the proprietary name Sutent®.

Sunitinib was first described in WO 2001/060814 and EP1255752 as one of a number of PK modulating compounds. The possibility of a number of salts is also disclosed therein. Such salts may include the hydrochloride, sulfate, carbonate, lactate, tartrate, malate, maleate and succinate salts. However, the disclosure is silent as to the nature of specific crystal forms of sunitinib.

It has long been an aim of the formulation scientist to develop alternative forms of active pharmaceutical ingredients (API). These forms, which include salts, solvates and hydrates, may be used as simple alternatives to the active ingredient for cost reasons or as a means of circumventing legal issues or they may possess advantageous properties such as an improved rate of dissolution, easier manufacture, increased bioavailability, decreased toxicity or increased efficacy. For the same reasons the formulation scientist would also seek to develop new polymorphs of an API.

Polymorphs are distinct solids sharing the same molecular formula, yet each polymorph may have distinct physical properties. Therefore a single compound may give rise to a variety of polymorphic forms where each form has different and distinct physical properties, such as different solubility profiles, different melting point temperatures and/or different X-ray diffraction peaks. The solubility of each polymorph may vary and consequently identifying the existence of polymorphs of an API is essential for providing pharmaceutical compositions with predictable solubility profiles. Polymorphic forms of a compound can be distinguished in a laboratory by X-ray diffraction spectroscopy and by other methods such as infrared spectrometry. Additionally, the properties of polymorphic forms of the same active pharmaceutical ingredient are well known in the pharmaceutical art to have an effect on the manufacture of drug product compositions comprising the API. For example, the solubility, stability, flowability, tractability and compressibility of the API as well as the safety and efficacy of drug product can be dependent on the polymorphic form.

The discovery of new polymorphic forms of a pharmaceutically useful compound provides an opportunity to improve the performance characteristics of a pharmaceutical product. It also adds to the material that a formulation scientist has available for designing, for example, a pharmaceutical dosage form of a drug with a targeted release profile or other desired characteristic. When a polymorphic form has been discovered to be useful and advantageous, either in the preparation of a pharmaceutical composition or as an intermediate in the preparation of another active pharmaceutical ingredient (API), the next challenge is to develop methods of synthesis that are simple and cost effective and provide the desired polymorph in the purest form possible.

Polymorphic form I of sunitinib appears to be disclosed, but in fact is not disclosed, in prior art patent application WO 2003/016305. According to example 1B of WO 2003/016305:
N-[2-(Diethylamino)ethyl]-5-[(5-fluoro-1,2-dihydro-2-oxo-3H-indol-3-ylidene)methyl]-2,4-dimethyl-1H-pyrrole-3-carboxamide may be purified by an aqueous pH=11 wash prior to formation of the L-malic acid salt. A solution of the free base in a mixture of 80:20 n-butanol:water (v:v) was prepared at 80° C. After cooling to 20° C. and stirring for 1 hour, significant crystallization was observed. A sample was analyzed by PXRD and found to be Crystal Form I. Filtration, drying, and co-milling of the crystals resulted in 99% yield.

However, it would appear to the skilled person reading this disclosure that in fact the method is missing a reference to the addition of malic acid and should properly relate to the preparation of sunitinib malate. This interpretation is consistent with the fact that crystalline forms of the free base are not mentioned anywhere in WO 2003/016305 apart from to highlight supposed disadvantages of the free base not being suitable for large scale preparation as an API. Indeed WO 2003/016305 solves this problem by preparing crystalline forms of sunitinib malate which are said to be suitable for large scale preparation. Further, the reference to “form I” is only used in WO 2003/016305 in relation to sunitinib malate form I.

Moreover, even if the skilled person were to view this disclosure as a method for preparing form I of sunitinib free base, the disclosure is not enabled as it would place an undue burden on the skilled person to determine which aqueous basic solutions could be used to purify the sunitinib. The skilled person would recognize that there are a plethora of possible basic solutions that could be used, but each may have distinct effects and cause the formation of a different crystalline form, or may cause stability or processing problems. WO 2003/016305 also highlights that sunitinib free base is not suitable for large scale manufacture of pharmaceutical products due to the fact that crystals of the free base may be crystallized as small particles. WO 2003/016305 further discloses that such small crystals are not desirable in large scale operations, it is preferable to have larger particle size crystals for ease in filtration. WO 2003/016305 solves the above problem by determining the properties of different sunitinib salts. The investigations resulted in the preparation of sunitinib malate which was claimed to resolve the above problems with processability.

### SUMMARY OF THE INVENTION

The inventors have surprisingly found that sunitinib in the free base form can be utilized in the preparation of pharmaceutical compositions and overcome the problems with large scale production disclosed in WO 2003/016305. Utilizing the free base as the API also has the advantage that an additional step of adding the salt forming acid and having to adjust parameters accordingly, such as buffering the pH, is not required and thus results in a simpler process.

In addition, counter-ions of salts can cause stability problems in pharmaceutical compositions and can cause problems with the formulation of said compositions. Further, particular counter-ions have been implicated in causing side effects in the patient population.

In view of the above comments, it is an object of the present invention to provide a novel crystalline, anhydrous form of sunitinib, designated form I, and processes for its preparation, and pharmaceutical compositions comprising it. The novel polymorph may be suitable for large scale production and may have other improved properties, such as improved solubility, bioavailability, stability including chemical and polymorphic stability, flowability, tractability, compressibility, compactability, toxicity, efficacy or safety.

Accordingly, in a first aspect of the present invention there is provided sunitinib form I having a characteristic XRPD spectrum comprising peaks (preferably major peaks) with 2θ values at 4.48 and 8.88±0.2 °2θ, when Cu α-radiation is used. Preferably the sunitinib form I has a characteristic XRPD spectrum comprising two or more peaks (preferably three or more, four or more, five or more, six or more, seven or more, eight or more, nine or more, ten or more, fifteen or more, twenty or more, or twenty five peaks) with 2θ values at 4.48, 7.07, 8.88, 10.57, 11.38, 12.78, 13.51, 14.95, 16.41, 18.86, 19.61, 20.58, 21.59, 22.53, 22.87, 23.09, 25.68, 27.22, 28.07, 29.19, 32.61, 34.09, 36.00, 41.93 and 44.00±0.2 °2θ, when Cu α-radiation is used. Preferably the sunitinib form I has a characteristic XRPD spectrum comprising peaks with 2θ values at 4.48, 7.07, 8.88, 10.57, 11.38, 12.78, 13.51, 14.95, 16.41, 18.86, 19.61, 20.58, 21.59, 22.53, 22.87, 23.09, 25.68, 27.22, 28.07, 29.19, 32.61, 34.09, 36.00, 41.93 and 44.00±0.2 °2θ, when Cu α-radiation is used. Preferably the sunitinib form I has an XRPD spectrum substantially as shown in FIG. 1.

Preferably the crystalline form I according to the first aspect of the invention is further characterized by a differential scanning calorimetry (DSC) with an endothermic peak at about 244° C., when a rate of heating of 10° C./min is used. Preferably the sunitinib form I has a DSC trace substantially as shown in FIG. 2.

In another embodiment, the crystalline form I according to the first aspect of the invention is further characterized by a thermogravimetric analysis (TGA) loss of 0% over a range of between about 25-220° C., when a rate of heating of 10° C./min is used. Preferably the sunitinib form I has a TGA trace substantially as shown in FIG. 3.

Preferably the crystalline form I of sunitinib according to the first aspect of the invention is anhydrous. In one embodiment the anhydrous form I comprises less than about 5%, more preferably less than about 4%, and most preferably less than about 2% water.

Preferably the crystalline form I of sunitinib has a chemical purity of greater than 99%, preferably greater than 99.3%, more preferably greater than 99.4%, even more preferably greater than 99.5%, yet more preferably greater than 99.6%, yet more preferably still greater than 99.7%, most preferably greater than 99.9%, preferably as measured by HPLC.

Preferably the crystalline form I of sunitinib has a polymorphic purity of greater than 98%, preferably greater than 99%, preferably greater than 99.3%, more preferably greater than 99.4%, even more preferably greater than 99.5%, yet more preferably greater than 99.6%, yet more preferably still greater than 99.7%, most preferably greater than 99.9%, preferably as measured by XRPD or DSC, preferably as measured by XRPD

According to a second aspect of the present invention there is provided a process for the preparation of crystalline form I of sunitinib, comprising the steps of:

(a) dissolving or suspending sunitinib in a solvent;
(b) causing crystalline form I of sunitinib to precipitate from the solution or suspension obtained in step (a); and
(c) isolating the solid form I obtained in step (b).

Preferably sunitinib is dissolved in step (a).

Preferably the solvent in step (a) is a hydroxylic solvent. Preferably the solvent comprises an alcohol, more preferably a C₁-C₆ alcohol. Preferred alcohols are alcohols R—OH, wherein R is C₁-C₆ alkyl, C₆-C₁₀ arylalkyl or C₆-C₁₀ aryl, each of which may optionally be substituted. Preferably R is unsubstituted C₁-C₆ alkyl. Preferably the alcohol is methanol, ethanol, n-propanol, isopropanol, n-butanol, sec-butanol, isobutanol, tert-butanol, or a mixture thereof. In one embodiment, the alcohol is not ethanol. Most preferably, the solvent is n-butanol. In alternative embodiments, the solvent further comprises water. In a particularly preferred embodiment, the solvent comprises an alcohol and water, preferably n-butanol and water, preferably in a n-butanol:water ratio of between about 60:40 to about 90:10, most preferably in a n-butanol:water ratio of about 80:20.

Typically, the solvent in step (a) is heated to dissolve the sunitinib. In preferred embodiments, when the solvent is n-butanol or is a solvent comprising n-butanol, preferably n-butanol and water, the temperature is between about 70-100° C., most preferably between about 95-98° C., or alternatively the solution is heated to reflux temperature.

In particularly preferred embodiments, the solution from step (a) is further filtered prior to step (b). Most preferably, the solution obtained in step (a) is filtered under vacuum or partial vacuum.

In another particularly preferred embodiment, step (b) comprises causing the solid to precipitate from the solution obtained in step (a) by cooling the solution, most preferably the solution is cooled to between about 0-5° C. Alternatively, in those embodiments where the solvent comprising sunitinib from step (a) has been heated to effect dissolution, the solution is cooled to ambient temperature, most preferably to between about 20-35° C. In said embodiments, the solution may also be cooled to below ambient temperature, for example to between about 0-20° C.

In some embodiments, the solvent in step (c) is allowed to evaporate to isolate the solid obtained in step (b) or in alternative preferred embodiments the solid precipitated in step (b) is isolated by filtration, preferably under vacuum. Preferably the isolated sunitinib is washed with the solvent utilized in step (a). Preferably the isolated sunitinib is allowed to dry until a constant weight is achieved, preferably at about 40° C., preferably under conditions of reduced pressure, most preferably under vacuum or partial vacuum.

Preferably the process of the second aspect of the present invention is carried out on an industrial scale, preferably to obtain sunitinib form I in batches of 0.1 kg or more, 0.5 kg or more, 1 kg or more, 5 kg or more, 10 kg or more, or 50 kg or more.

Preferably the sunitinib form I is obtained in a yield of 50% or more, 60% or more, 70% or more, 80% or more, or 90% or more.

Preferably the sunitinib form I obtained has a chemical purity of 99% or more, 99.3% or more, 99.4% or more, 99.5% or more, 99.6% or more, 99.7% or more, or 99.9% or more, preferably as measured by HPLC.

Preferably the sunitinib form I obtained has a polymorphic purity of 98% or more, 99% or more, 99.3% or more, 99.4% or more, 99.5% or more, 99.6% or more, 99.7% or more, or 99.9% or more, preferably as measured by XRPD or DSC, preferably as measured by XRPD.

A third aspect of the present invention provides a process for preparing sunitinib malate, comprising reacting the sunitinib form I according to the first aspect of the invention or prepared by a process according to the second aspect of the invention with malic acid. Preferably the malic acid is L-malic acid, alternatively the malic acid is D-malic acid.

Preferably the sunitinib form I according to the first aspect of the invention or prepared by a process according to the second aspect of the invention, or the sunitinib malate prepared by a process according to the third aspect of the invention, is suitable for use in medicine, preferably for treating or preventing cancer or a tumor, more preferably for treating or preventing unresectable and/or metastatic malignant gastrointestinal stromal tumor (GIST) or advanced and/or metastatic renal cell carcinoma (MRCC).

According to a fourth aspect of the present invention there is provided a pharmaceutical composition comprising the sunitinib form I according to the first aspect of the invention or prepared by a process according to the second aspect of the invention or the sunitinib malate prepared by a process according to the third aspect of the invention. Preferably the pharmaceutical composition according to the fourth aspect of the invention is for use in the treatment or prevention of cancer and/or tumors. More preferably, the use is the treatment or prevention of unresectable and/or metastatic malignant gastrointestinal stromal tumor (GIST) or advanced and/or metastatic renal cell carcinoma (MRCC).

According to a fifth aspect of the present invention there is provided the use of the sunitinib form I according to the first aspect of the invention or prepared by a process according to the second aspect of the invention, or the use of the sunitinib malate prepared by a process according to the third aspect of the invention, for the manufacture of a medicament for treating or preventing cancer or a tumor, preferably for the manufacture of a medicament for treating or preventing unresectable and/or metastatic malignant gastrointestinal stromal tumor (GIST) or advanced and/or metastatic renal cell carcinoma (MRCC).

According to a sixth aspect of the present invention there is provided a method of treating or preventing cancer or a tumor, the method comprising administering to a patient in need thereof a therapeutically or prophylactically effective amount of the sunitinib form I according to the first aspect of the invention or prepared by a process according to the second aspect of the invention, or a therapeutically or prophylactically effective amount of the sunitinib malate prepared by a process according to the third aspect of the invention, or a therapeutically or prophylactically effective amount of a pharmaceutical composition according to the fourth aspect of the invention. Preferably the method is for treating or preventing unresectable and/or metastatic malignant gastrointestinal stromal tumor (GIST) or advanced and/or metastatic renal cell carcinoma (MRCC). Preferably the patient is a mammal, preferably a human.

### BRIEF DESCRIPTION OF THE ACCOMPANYING FIGURES

FIG. 1 describes the X-ray powder diffraction (XRPD) of sunitinib form I according to the invention.

FIG. 2 describes the differential scanning calorimetry (DSC) of sunitinib form I according to the invention.

FIG. 3 describes the thermogravimetric analysis (TGA) of sunitinib form I according to the invention.

### DETAILED DESCRIPTION OF THE INVENTION

As outlined above, the present invention provides a novel crystalline form of sunitinib base and processes for its preparation. The crystalline form according to the invention can be used in the preparation of sunitinib malate or other sunitinib salts or polymorphs, or as an API in a pharmaceutical product. Preferred embodiments of the process for the preparation of sunitinib form I are described below.

A preferred embodiment of a process for the preparation of crystalline form I of sunitinib comprises the steps of:

(a) dissolving sunitinib in a solvent;
(b) causing crystalline form I of sunitinib to precipitate from the solution obtained in step (a); and
(c) isolating the crystalline sunitinib obtained in step (b).

Preferably the solvent used is an alcohol, more preferably a C₁-C₆ alcohol. Most preferably, the solvent is n-butanol. In alternative embodiments, the solvent further comprises water.

In a particularly preferred embodiment, the solvent comprises n-butanol and water, preferably in a ratio of between about 60:40 to about 90:10, most preferably in a ratio of about 80:20 n-butanol:water.

In preferred processes, complete dissolution of the sunitinib is indicated when a clear solution from step (a) is obtained. The clear solution is in some embodiments obtained by dissolving the sunitinib in the relevant solvent by heating. Preferably, when the solvent is n-butanol or when the solvent comprises n-butanol, the solvent is heated at between about 60-100° C. The inventors have found that heating to between about 70-100° C., most preferably to between about 95-98° C., or alternatively to reflux temperature is most advantageous. It is within the skill set of the skilled person to determine the reflux temperatures of the preferred solvents. It will also be apparent that the sunitinib referred to according to the invention is sunitinib free base.

The solution, however obtained, may preferably be filtered at this stage in order to further remove particulate impurities that may be present. This has been found to result in an even purer final product by removing any particulate matter that may act as seed material for polymorphic impurities.

Causing the crystalline sunitinib according to the invention to precipitate as required by step (b) can be achieved in any of a number of ways by the skilled person.

The inventors have found that cooling the solution will cause the desired crystalline form to precipitate from the solution. The skilled person will realize that the solution can be cooled to ambient temperature when the solution has been heated to effect dissolution of the sunitinib or indeed below that. The inventors have found that cooling to between about 0-20° C., preferably to between about 0-10° C., most preferably to between about 0-5° C., is particularly advantageous. Crystalline form I may also be caused to precipitate for example by stirring the solution or by cooling the solution, even in those embodiments wherein dissolution of the sunitinib was not effected by heating, to below ambient temperature, preferably to between about 0-10° C., most preferably to between about 0-5° C. A combination of stirring and allowing the solution to cool can also be employed. Stirring of the solution to effect precipitation may also be employed in those embodiments wherein the solution has been heated to effect dissolution. In these embodiments, it is envisaged that the stirring will be carried out during cooling or indeed once the solution has cooled. In any case, the stirring conditions may be varied and still remain within the scope of the invention.

The solid crystalline product obtained can then be isolated by any means common in the field or known to the skilled artisan. Preferably the solid obtained is washed with the same solvent as utilized in step (a). Thus, for example when the solvent used is n-butanol, it is preferred that the solid is washed with n-butanol, or when an 80:20 mixture of n-butanol:water is used as the solvent, the same is used to wash the solid obtained. In one embodiment, the solid is obtained by evaporation of the solvent under ambient conditions. However, in a particularly preferred embodiment, the solid product is filtered and dried. Preferably the product is dried at a temperature that does not induce conversion of the crystalline form or causes the resultant crystalline form to degrade. The inventors have found that drying the product at between about 30-50° C., preferably at about 40° C., is advantageous. Preferably, in certain embodiments, the solid product is dried under vacuum or partial vacuum, most preferably at about 40° C. until a constant weight is obtained.

The process of the invention provides sunitinib form I in a particularly pure form. In certain embodiments, there is provided sunitinib form I having a chemical purity of greater than 99%, preferably greater than 99.3%, more preferably greater then 99.4%, even more preferably greater than 99.5%, yet more preferably greater than 99.6%, yet more preferably still greater than 99.7%, most preferably greater than 99.9%, preferably as measured by HPLC.

As mentioned previously the sunitinib form I according to the invention may be used as an intermediate in the preparation of salts of sunitinib. Non-limiting examples include the hydrochloride, sulfate, carbonate, lactate, tartrate, malate, maleate or succinate salts. The salts may be prepared in any way known to the skilled person, but generally preparation of the salts involves contacting the sunitinib form I according to the invention with an appropriate acid. In particularly preferred embodiments, the acid is malic acid, but in alternative embodiments the salt prepared may be any pharmaceutically acceptable salt or indeed any salt useful in the preparation of a pharmaceutically acceptable form of sunitinib.

The sunitinib form I may also be useful in the preparation of advantageous hydrates and solvates, by contacting the sunitinib form I according to the invention with water/aqueous solvent or a desired solvent respectively under appropriate conditions. The preparation of said hydrates, solvates and salts is well within the skill set of the skilled person to achieve and should be considered to be within the scope of the invention. It is also envisaged that the sunitinib form I according to the invention may be used as an intermediate in the preparation of other polymorphic forms. For example, WO 2003/016305 discloses methods for the preparation of the malate salt of sunitinib; the disclosure is incorporated herein by reference.

Accordingly, there is provided a process for preparing sunitinib malate, comprising reacting sunitinib form I according to the invention with malic acid. In a particularly preferred embodiment, the malic acid is L-malic acid or alternatively is D-malic acid.

As alluded to above, the apparent disclosure of sunitinib form I in WO 2003/016305 appears to be a mistake and in fact relates to the preparation of sunitinib malate form I. The inventors followed the method disclosed in WO 2003/016305, but added malic acid to prepare sunitinib malate. The resultant XRP diffractogram is the same as that reported in WO 2003/016305 for form I sunitinib malate, showing that indeed the disclosure was meant to provide a method for preparing sunitinib malate.

A further aspect of the invention provides a composition comprising crystalline sunitinib according to the invention or prepared according to the invention and further comprising one or more pharmaceutically acceptable excipient(s).

In a preferred embodiment, a pharmaceutical composition is provided comprising sunitinib malate prepared according to the invention. Further preferred embodiments provide a pharmaceutical composition comprising sunitinib malate prepared according to the invention for use in the treatment or prevention of cancer and/or tumors, preferably for the treatment or prevention of unresectable and/or metastatic malignant gastrointestinal stromal tumor (GIST) or advanced and/or metastatic renal cell carcinoma (MRCC).

The pharmaceutical composition according to the invention can be a solution or suspension, but is preferably a solid oral dosage form. Preferred oral dosage forms in accordance with the invention include tablets, capsules and the like which, optionally, may be coated if desired. Tablets can be prepared by conventional techniques, including direct compression, wet granulation and dry granulation. Capsules are generally formed from a gelatin material and can include a conventionally prepared granulate of excipients in accordance with the invention.

The pharmaceutical composition according to the present invention typically comprises one or more conventional pharmaceutically acceptable excipient(s) selected from the group comprising a filler, a binder, a disintegrant, a lubricant, and optionally further comprises at least one excipient selected from coloring agents, adsorbents, surfactants, film formers and plasticizers.

If the solid pharmaceutical formulation is in the form of coated tablets, the coating may be prepared from at least one film former such as hydroxypropyl methyl cellulose, hydroxypropyl cellulose or methacrylate polymers which optionally may contain at least one plasticizer such as polyethylene glycols, dibutyl sebacate, triethyl citrate, and other pharmaceutical auxiliary substances conventional for film coatings, such as pigments, fillers and others.

Preferably the pharmaceutical compositions according to one aspect of the invention are for use in treating or preventing disorders related to abnormal protein kinase (PK) activity. Such diseases include, but are not limited to, diabetes, hepatic cirrhosis, cardiovascular disease such as atherosclerosis, angiogenesis, immunological disease such as autoimmune disease, malignant gastrointestinal stromal tumor (MGIST) and metastatic renal cell carcinoma (MRCC).

The details of the invention, its objects and advantages are illustrated below in greater detail by non-limiting examples.

### EXAMPLES

### Example 1

### Preparation of Sunitinib Form I According to the Invention

Sunitinib (1 eq) was dissolved in n-butanol:water (80:20) (v/v) (10 vol) at 85-90° C. to obtain a clear solution. The hot solution was filtered through a Buchner funnel under vacuum. The filtrate was cooled to ambient temperature between about 22-27° C., and a yellow to orange solid was obtained. The solid thus obtained was filtered using a Buchner funnel under vacuum and washed with n-butanol:water (80:20) (v/v). The solid was then dried under vacuum at about 40° C. for 3 hours to obtain sunitinib crystal form I.

### % Yield=92%

HPLC purity=98.24%

### Example 2

### Preparation of Sunitinib Form I According to the Invention

Sunitinib (1 eq) was dissolved in n-butanol (10 vol) at 95-98° C. to obtain a clear solution. The hot solution was filtered through a Buchner funnel under vacuum. The filtrate was cooled to ambient temperature between about 22-27° C., and a yellow to orange solid was obtained. The solid thus obtained was filtered using a Buchner funnel under vacuum and washed with n-butanol. The solid was then dried under vacuum at about 40° C. for 3 hours to obtain sunitinib crystal form I.

### % Yield=85%

HPLC purity=99.34%

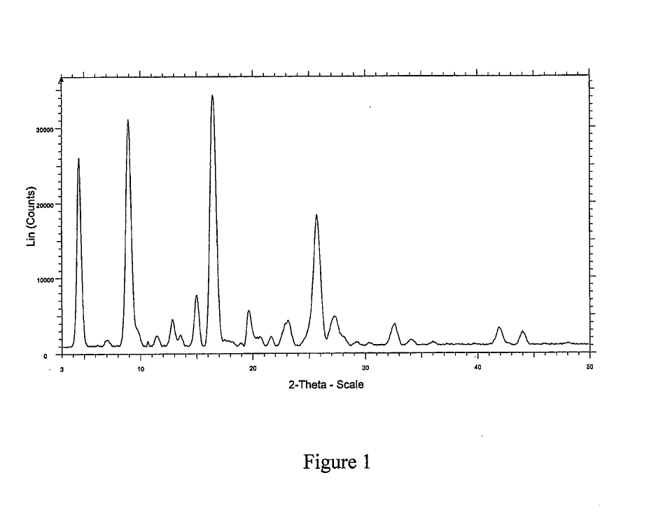

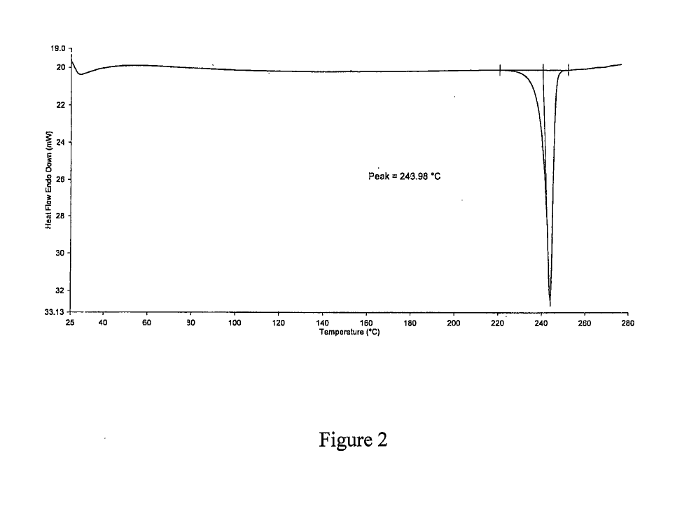

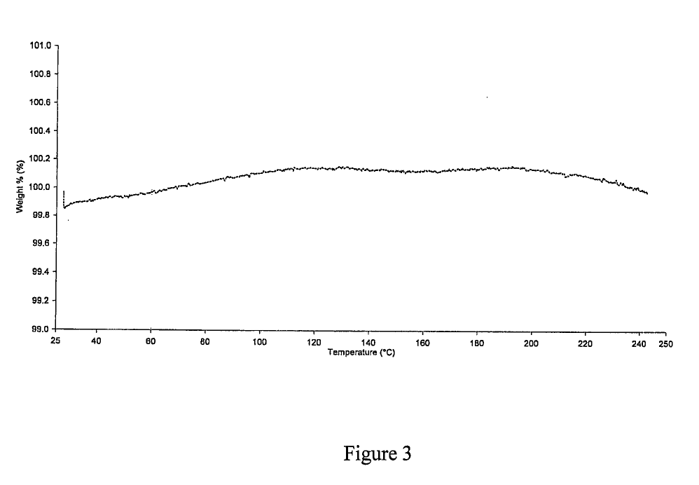

The resultant solids obtained from the examples were characterized by XRPD (shown in FIG. 1), DSC (shown in FIG. 2) and TGA (shown in FIG. 3), which confirmed that sunitinib form I was prepared by the processes according to the invention.

The XRPDs were recorded on a Bruker D8 Advance Instrument, using Cu α-radiation as the X-ray source, with a 20 range of from 3 to 50°, a step-size of 0.5° and a time/step of 1 sec.

XRPD: 4.48, 7.07, 8.88, 10.57, 11.38, 12.78, 13.51, 14.95, 16.41, 18.86, 19.61, 20.58, 21.59, 22.53, 22.87, 23.09, 25.68, 27.22, 28.07, 29.19, 32.61, 34.09, 36.00, 41.93, and 44.00.

The DSCs were recorded on a Perkin Elmer Pyris 6, with a temperature range of from 25° C. to 280° C. and a rate of heating of 10° C./min.

DSC peak: 244° C.

The TGAs were recorded on a Perkin Elmer Pyris 6, with a temperature range of from 25° C. to 250° C. and a rate of heating of 10° C./min.

It will be understood that the present invention has been described above by way of example only. The examples are not intended to limit the scope of the invention. Various modifications and embodiments can be made without departing from the scope and spirit of the invention, which is defined by the following claims only.

1.-40. (canceled)

41. Sunitinib form I having a characteristic XRPD spectrum comprising peaks with 2θ values at 4.48 and 8.88±0.2 °2θ.

42. Sunitinib form I according to claim 41:
(i) having a characteristic XRPD spectrum comprising two or more peaks with 2θ values at 4.48, 7.07, 8.88, 10.57, 11.38, 12.78, 13.51, 14.95, 16.41, 18.86, 19.61, 20.58, 21.59, 22.53, 22.87, 23.09, 25.68, 27.22, 28.07, 29.19, 32.61, 34.09, 36.00, 41.93 and 44.00±0.2 °2θ; and/or
(ii) having an XRPD spectrum substantially as shown in FIG. 1; and/or
(iii) characterized by a differential scanning calorimetry (DSC) with an endothermic peak at about 244° C.; and/or
(iv) characterized by a thermogravimetric analysis (TGA) loss of 0% over a range of between about 25-220° C.

43. Sunitinib form I according to claim 41, having an HPLC purity of greater than 99%.

44. A process for the preparation of crystalline form I of sunitinib according to claim 41, comprising the steps of:
(a) dissolving sunitinib in a solvent;
(b) causing crystalline form I of sunitinib to precipitate from the solution obtained in step (a); and
(c) isolating the solid form I obtained in step (b).

45. A process according to claim 44, wherein the solvent in step (a):
(i) is a hydroxylic solvent; and/or
(ii) comprises an alcohol; and/or
(iii) is n-butanol; and/or
(iv) further comprises water; and/or
(v) comprises n-butanol and water; and/or
(vi) comprises n-butanol and water in a ratio of between about 60:40 to about 90:10; and/or
(vii) comprises n-butanol and water in a ratio of about 80:20; and/or
(viii) is heated to dissolve the sunitinib; and/or
(ix) comprises n-butanol and is heated to between about 70-100° C.; and/or
(x) comprises n-butanol and is heated to between about 95-98° C.

46. A process according to claim 44, wherein the solution obtained in step (a) is filtered prior to step (b).

47. A process according to claim 44, wherein:
(i) the crystalline form I of sunitinib is caused to precipitate from the solution obtained in step (a) by cooling the solution; and/or
(ii) the crystalline form I of sunitinib is caused to precipitate from the solution obtained in step (a) by cooling the solution to between about 0-5° C.; and/or
(iii) when the solvent has been heated in step (a) to effect dissolution of the sunitinib, the crystalline form I of sunitinib is caused to precipitate from the solution obtained in step (a) by cooling the solution to between about 20-35° C.

48. A process according to claim 44, wherein in step (c):
(i) the solid obtained in step (b) is isolated by filtration; and/or
(ii) the isolated solid is washed with the solvent utilized in step (a); and/or
(iii) the isolated solid is dried until a constant weight is achieved; and/or
(iv) the isolated solid is dried at about 40° C. under conditions of reduced pressure until a constant weight is achieved.

49. A process for preparing sunitinib malate, comprising reacting sunitinib form I according to claim 41 with malic acid.

50. A process according to claim 49, wherein the malic acid is L-malic acid or D-malic acid.

51. A pharmaceutical composition comprising sunitinib form I according to claim 41 and one or more pharmaceutically acceptable excipients.

52. A method of treating or preventing cancer or a tumor, the method comprising administering to a patient in need thereof a therapeutically or prophylactically effective amount of sunitinib form I according to claim 41.

53. A method of treating or preventing cancer or a tumor, the method comprising administering to a patient in need thereof a therapeutically or prophylactically effective amount of a pharmaceutical composition according to claim 51.

54. A method of treating or preventing unresectable and/or metastatic malignant gastrointestinal stromal tumor (GIST) or advanced and/or metastatic renal cell carcinoma (MRCC), the method comprising administering to a patient in need thereof a therapeutically or prophylactically effective amount of sunitinib form I according to claim 41.

55. A method of treating or preventing unresectable and/or metastatic malignant gastrointestinal stromal tumor (GIST) or advanced and/or metastatic renal cell carcinoma (MRCC), the method comprising administering to a patient in need thereof a therapeutically or prophylactically effective amount of a pharmaceutical composition according to claim 51.

56. A method according to claim 52, wherein the patient is a mammal such as a human.

57. A method according to claim 53, wherein the patient is a mammal such as a human.

58. A method according to claim 54, wherein the patient is a mammal such as a human.

59. A method according to claim 55, wherein the patient is a mammal such as a human.
